(19) United States
(12) Patent Application Publication
(10) Pub. No.: US 2008/0166791 A1
(43) Pub. Date: Jul. 10, 2008

(54) Universal glucometer/glucovial connector

(75) Inventors: Steven Kim, Palisades Park, NJ (US); Douglas Kim, Hillsborough, NJ (US); Tae H. Kim, Little Ferry, NJ (US)

Correspondence Address: SEYFARTH SHAW, LLP, 815 CONNECTICUT AVENUE, N.W., SUITE 500, WASHINGTON, DC, 20006, US

(73) Assignee: HealthPia America, Palisades Park, NJ (US)

(21) Appl. No.: 12/007,230

(22) Filed: Jan. 8, 2008

Related U.S. Application Data
(60) Provisional application No. 60/879,039, filed on Jan. 8, 2007.

(51) Int. Cl. C12M 1/00

(52) U.S. Cl. 435/287.1

(57) ABSTRACT

A universal glucometer connector system comprising: a glucometer for measuring glucose concentration of blood, and having a top, bottom and side surface; a jacket for retaining the glucometer; a first opening in the jacket for receiving a glucovial that can be one of a plurality of shapes and sizes.

This invention claims the benefit of the Provisional Patent Application 60/879,039 filed with the U.S. Patent and Trademark Office on Jan. 8, 2007, which is hereby incorporated by reference in its entirety.

### BACKGROUND OF THE INVENTION

The embodiments of the invention relate to a universal connector for medical equipment and more particularly to a universal connector included in a glucometer/glucovial system. Although embodiments of the invention are suitable for a wide scope of applications, they are particularly suitable for retaining a “glucometer,” or a device that measures blood glucose (sugar) levels, that is adaptable for use with a mobile phone or other mobile technologies.

Diabetes mellitus is a pervasive, incurable metabolic disorder characterized primarily by hyperglycemia, otherwise known as high blood sugar. The World Health Organization estimated that at least 171 million people worldwide suffered from the disease in 1996 and its incidence is expected to continue to grow rapidly in the coming decades. The Centers for Disease Control and Prevention estimate that 1 in 3 Americans born after 2000 will develop diabetes in his/her lifetime.

A person with diabetes must monitor blood glucose concentration periodically so as to appropriately adjust treatment and/or diet. Methods for monitoring blood glucose concentration generally involve drawing blood stamples from the fingers by using a needle or a pricking device. The blood is then provided to a diagnostic device, such as a “glucometer,” that measures the blood glucose concentration and outputs a reading to the individual. This monitoring procedure can be performed at home or during a visit to a doctor's office. However, many people with diabetes are highly active and prefer to monitor their blood glucose concentration irrespective of where they are located. Therefore, there is substantial use of light-weight, robust and portable glucometers.

Such portable glucometers can be based on a variety of analytic techniques, including electrochemical or spectroscopic techniques. Several examples of the latter are: U.S. Pat. No. 6,241,663 to Wu et al. and U.S. Pat. No. 5,551,422 to Simonsen et al. The portable glucometers generally display the results of the blood glucose analysis to an LCD display or some other kind of local read-out mechanism that can be read by the user. This read-out mechanism need not be local and there are advantages to using remote read-out devices via wireless devices, such as a mobile phone shown in U.S. Pat. Appl. Pub. 20040133455. Wireless blood glucose concentration analyzing devices allow the transmission of data to healthcare professionals who may collect the data for diagnostic purposes. Regardless of the analysis technique a storage mechanism is used to store test strips for determining blood glucose concentration.

Generally, the storage mechanism is a plastic container or a vial referred to as a glucovial. If the storage environment for the test strips inside the glucovial is not free of dust, humidity or other contaminants the test strips may become compromised. Use of compromised test strips may damage the glucometer or result in erroneous readings leading to treatments that may have the potential to damage the health of the user. Because of the need for accurate readings of blood glucose concentration, the integrity of the storage mechanism is critical to the proper functioning of the glucometer system so as to monitor diabetes. The negative effect on the performance of test strips due to adverse storage conditions has been the subject of recent studies (see, for example: R. Bamberg et al., “Effect of adverse storage conditions on performance of glucometer test strips”, Clin Lab Sci. 2005 Fall; 18(4):203-9).

### SUMMARY OF THE INVENTION

Accordingly, embodiments of the invention are directed to a universal glucometer/glucovial connector that substantially obviates one or more of the problems due to limitations and disadvantages of the related art.

An object of embodiments of the invention is to retain a glucometer with a universal connector.

Another object of embodiments of the invention to retain a glucometer and a glucovial with a universal connector.

Another object of embodiments of the invention is to retain a glucometer and a glucovial in a storage system including a holster.

Additional features and advantages of embodiments of the invention will be set forth in the description which follows, and in part will be apparent from the description, or may be learned by practice of embodiments of the invention. The objectives and other advantages of the embodiments of the invention will be realized and attained by the structure particularly pointed out in the written description and claims hereof as well as the appended drawings.

To achieve these and other advantages and in accordance with the purpose of embodiments of the invention, as embodied and broadly described, the universal glucometer/glucovial connector is included as a jacket for retaining the glucometer in a universal glucometer connector system including: a glucometer for measuring glucose concentration of blood, and having a top, bottom and side surface; a first opening in the jacket for receiving a glucovial that can be one of a plurality of shapes and sizes.

In another aspect, the universal glucometer/glucovial connector is included as a jacket for retaining the glucometer in a universal glucometer connector system including: a glucometer for measuring glucose concentration of blood; a glucovial having a container for receiving test strips and a cap for sealing the container; a first opening in the jacket such that the glucometer can receive test strips; and a second opening in the jacket that surrounds the glucovial.

In another aspect, the universal glucometer/glucovial connector is included as a jacket for receiving a glucovial that can be one of a plurality of shapes and sizes in a universal glucometer connector system including: a glucometer for measuring glucose concentration of blood; a mobile phone for receiving signals from the glucometer, interpreting the signals and displaying the data including the measured glucose concentration.

In a yet another aspect, the universal glucometer/glucovial connector is included as a jacket for receiving a glucovial in a universal glucometer connector system comprising: a storage system for a universal glucometer connector system comprising: a glucometer for measuring glucose concentration of blood; a holster that has a top portion and a main-body portion, wherein the main-body portion includes: an outward face having a recessed portion in which the glucometer can be reversibly fixed and a second portion where the mobile phone can be reversibly fixed; an inward face opposite to the outward face and having a clip that allows the holster to be fixed to an article of clothing; and the top portion extends above the clip to form a post on which the jacket can be stored.

It is to be understood that both the foregoing general description and the following detailed description are exemplary and explanatory and are intended to provide further explanation of embodiments of the invention as claimed.

### BRIEF DESCRIPTION OF THE DRAWINGS

The accompanying drawings, which are included to provide a further understanding of embodiments of the invention and are incorporated in and constitute a part of this specification, illustrate embodiments of the invention and together with the description serve to explain the principles of embodiments of the invention.

FIG. 1a shows a top view of a glucometer;

FIG. 1b shows a side view of the glucometer in FIG. 1a;

FIG. 1c shows a bottom view of the glucometer in FIG. 1a;

FIG. 1d shows a top view of a mobile phone that can be used with the glucometer in FIG. 1a;

FIG. 2a shows an exploded view of a glucovial with a cylindrical shape;

FIG. 2b shows an assembly view of the glucovial shown in FIG. 2a;

FIG. 3a shows encompassing a glucometer with a glucometer encompassing jacket;

FIG. 3b shows connecting a jacketed glucometer to a glucovial;

FIG. 3c shows an assembly of the jacketed glucometer and glucovial;

FIG. 4a shows a top view of a glucometer attaching jacket;

FIG. 4b shows a bottom view of the glucometer attaching jacket shown in FIG. 4a;

FIG. 5a shows attaching the glucometer attaching jacket shown in FIG. 4a and FIG. 4b to a glucovial;

FIG. 5b shows attaching a glucometer to the glucometer attaching jacket attached to the glucovial;

FIG. 5c shows an assembly of a jacketed glucometer glucovial assembly;

FIG. 6a shows a cylindrical glucovial with a circular cross-section having a diameter D2 that is approximately equal to the width D1 of a glucometer;

FIG. 6b shows a jacketed glucometer assembly, in which a glucometer is encompassed in a glucometer encompassing jacket, which is attached to the cylindrical glucovial shown in FIG. 6a;

FIG. 6c shows a glucometer attached to a glucometer attaching jacket, which is attached to the cylindrical glucovial shown in FIG. 6a;

FIG. 7a shows an oversized cylindrical glucovial with a circular cross-section having a diameter D3 that is substantially larger than the width D1 of a glucometer;

FIG. 7b shows a jacketed glucometer assembly, in which a glucometer is encompassed in a glucometer encompassing jacket 30, which is attached to the oversized cylindrical glucovial shown in FIG. 7a;

FIG. 7c shows a glucometer attached to a glucometer attaching jacket, which is attached to the oversized cylindrical glucovial shown in FIG. 7a;

FIG. 8a shows a square glucovial with a square cross-section;

FIG. 8b shows a jacketed glucometer assembly, in which a glucometer is encompassed in a glucometer encompassing jacket, which is attached to the square glucovial shown in FIG. 8a;

FIG. 8c shows a glucometer affixed to a glucometer attaching jacket, which is attached to the glucovial shown in FIG. 8a;

FIG. 9a shows a side view of storing a jacket and glucometer in a holster;

FIG. 9b shows a front view of storing a jacket and glucometer in a holster;

FIG. 10a shows a side view of a jacket and glucometer stored in a holster and the placement of a mobile phone on the holster;

FIG. 10b shows a front view of a jacket and glucometer stored in a holster;

### DETAILED DESCRIPTION OF THE PREFERRED EMBODIMENTS

Reference will now be made in detail to the preferred embodiments of the invention, examples of which are illustrated in the accompanying drawings. It will be apparent to those skilled in the art that various modifications and variations can be made in the embodiments of the invention, the universal glucometer/glucovial, without departing from its spirit or scope. Thus, it is intended that embodiments of the invention cover the modifications and variations of this invention provided they come within the scope of the appended claims and their equivalents. In the drawings, the thicknesses of layers and regions are exaggerated for clarity. Like reference numerals in the drawings denote like elements.

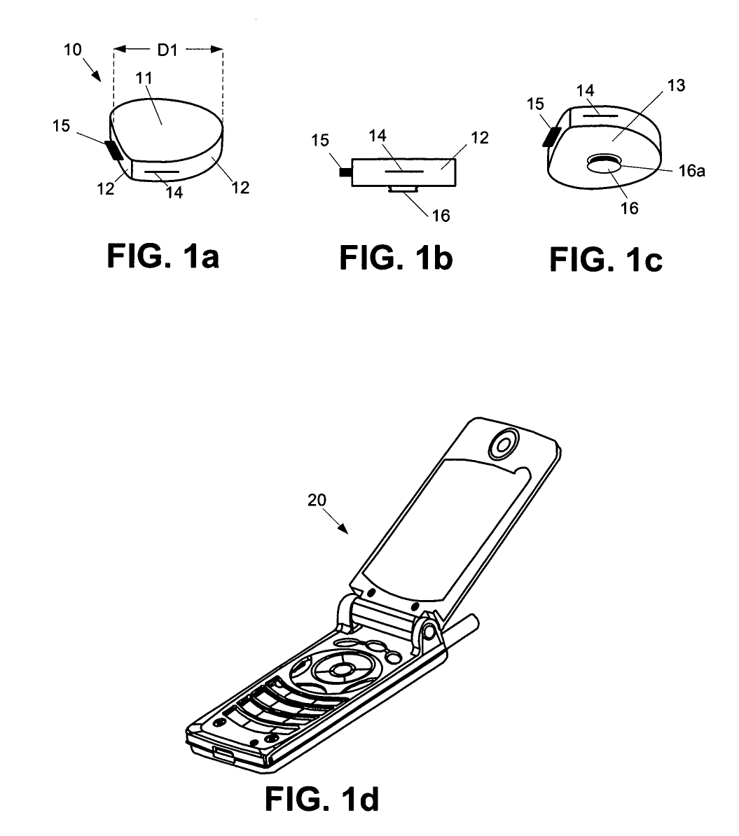

FIGS. 1a-1c show three views of a glucometer 10 that can be used in a universal glucometer connector system in accordance with embodiments of the invention. FIG. 1d shows a top view of a mobile phone that can be used with the glucometer in FIG. 1a. The glucometer 10 is portable and powered by either disposable or rechargeable batteries. The glucometer 10 is used with another device, such as a mobile phone 20 shown in FIG. 1d, that has an LCD display for displaying the results of a blood glucose concentration analysis from the glucometer 10. The glucometer 10 can also be used with other devices having similar display capabilities, such as a portable computer. Alternatively, the glucometer 10 can have a display, such as a digital read-out on an LCD display and/or a series of light emitting diodes. Li ion batteries are typically used to power the glucometer 10, although other types of batteries can alternatively be used.

FIG. 1a shows a top view of a glucometer; FIG. 1b shows a side view of the glucometer in FIG. 1a; and FIG. 1c shows the bottom view of the glucometer in FIG. 1a. As shown in FIG. 1a, the glucometer 10 has a top surface 11 with an edge bordering the side surface 12 of the glucometer 10, which is oriented perpendicularly to the top surface 11 of the glucometer 10. The glucometer 10 also has a width D1 across the top surface 11.

As shown in FIG. 1c, the glucometer 10 is has a bottom surface 13 that also has an edge bordering the side surface 12 of the glucometer 10. The bottom surface 13 can be parallel to the top surface 11. The side surface 12 has a test strip receiving slot 14 for receiving test strips (not shown). The test strips can have contacts and/or circuitry that connect to the glucometer 10 so as to analyze blood deposited on the test strip by the user to determine glucose concentration in the blood. For example, a user places 1-2 drops of blood on a single test strip and inserts the blood-coated test strip into test strip receiving slot 14. Electrodes (not shown) on the interior of the glucometer 10 contact to the test strip so as to pass an electrical current through the test strip and blood to measure blood glucose concentration. Blood glucose concentration data is determined by measuring the charge passing through the electrode. In the alternative, blood glucose concentration data can be derived from a photoabsorption or spectroscopic analysis. Subsequently, the blood glucose concentration data is processed by electronics (not shown) within the glucometer 10, then outputted to the user through a mobile phone or some other device external to the glucometer 10. The test strip is then removed from the glucometer 10 and discarded.

As shown in FIG. 1b, the glucometer 10 has a data output connector 15 for connecting the glucometer 10 to the mobile phone (not shown) so as to transfer data, such as the blood glucose concentration data described above. The data output connector 15 can be located on a side surface 12 of the glucometer 10, as shown in FIG. 1a, or on another surface of the glucometer 10. The data output connector 15 is a serial connection, such as a USB connection. The serial connection can also be a wireless connection, such as wi-fi, local wireless network or a wireless USB connection. The wireless connection can be made one of a plurality of processing devices including: a mobile phone, a personal digital assistant, a personal computer or a digital music player.

FIG. 1c shows the bottom surface 13 of the glucometer 10 and the glucometer fastening knob 16. The glucometer fastening knob 16, shown in FIGS. 1b and 1c, is used as a mechanism for attaching the glucometer 10 to a clip, carrying case or holster for storage or transport. The glucometer fastening knob 16 is configured to snap-fasten into a glucometer fastening knob receiving hole (not shown). As shown in FIG. 1c, the glucometer fastening knob 16 can have a lip 16a that snap-fastens into a glucometer fastening knob receiving hole constructed with a corresponding mating lip.

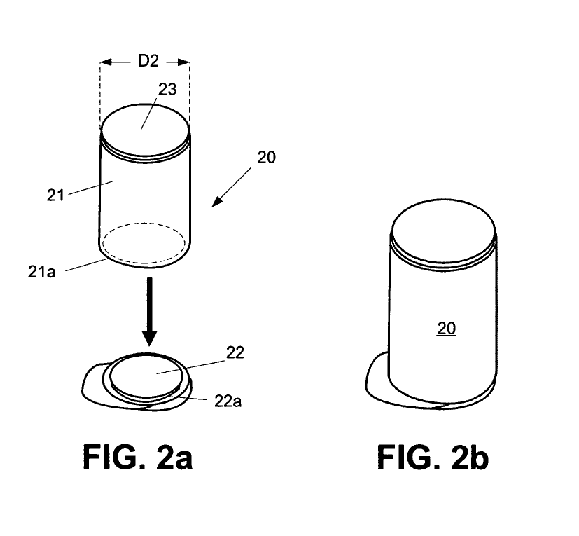

FIG. 2a shows an exploded view of a glucovial with a cylindrical shape and FIG. 2b shows an assembly view of the glucovial shown in FIG. 2a. As shown in FIG. 2a, the glucovial 20 includes a glucovial main body 21 having a hollow cavity and a glucovial cap 22 for enclosing the hollow cavity. The glucovial main body 21 has a cap mating flange 21a and the glucovial cap 22 has a main body mating flange 22a that contact each other when the glucovial cap 22 is attached to the glucovial main body 21 as shown in FIG. 2a. The cap mating flange 21a and the main body mating flange 22a can be constructed such that they snap-fasten to each other to seal the glucovial 20.

Sealing the glucovial 20 using the glucovial cap 22 keeps the storage environment for the test strips inside the glucovial main body 21 free of contaminants, such as dust and moisture. The glucovial main body 21 has a glucovial top 23 located on the opposite end of the glucovial main body 21 from the cap mating flange 21a, as shown in FIG. 2a. Test strips (not shown) used in the analysis of blood or other fluids are placed within the hollow cavity of the glucovial main body 21. The hollow cavity of the glucovial 20 inside the glucovial main body 21 can also contain a chemical desiccant or other device for removing moisture from the interior of the glucovial 20. Suitable chemical desiccants include silica gel, calcium sulfate. FIG. 2b shows the glucovial 20 sealed with the glucovial cap 22 such that the cap mating flange 21a and the main body mating flange 22a are snap-contacted with each other.

Although the glucovial 20 shown in FIGS. 2a and 2b is cylindrical and has a circular cross-section, the glucovial 20 can have one of a plurality of shapes and sizes. The cross-section of the glucovial 20 can, for example, be rectangular, square or triangular. Further, the glucovial 20 can have a shape other than a cylindrical shape. For example, the glucovial 20 can have a spherical or a box shape. The diameter D2 of the circular cross-section of the glucovial 20 can be larger, smaller or substantially equal to the diameter D1 of the glucometer 10.

The glucovial main body 21 and glucovial 22 are composed of a rigid material to protect the test strips, such as plastic or polymeric materials. The glucovial main body 21 can contain internal structures suited for storing test strips (not shown). Such internal structures in the glucovial main body 21 include plastic guide rails to facilitate loading and unloading of test strips, a catch mechanism at the bottom of the glucovial preventing the test strips from falling out of the glucovial 20 or a spring-loaded test strip delivery system that allows the test strips to be removed sequentially from the glucovial 20. The flange section of the glucovial 20 (not shown), where the glucovial cap 22 contacts and seals with the glucovial main body 21, can be reinforced by a different material that is a less brittle plastic to prevent damage due to repeated opening and closing. Further, a flexible plastic strip (not shown) can be used to connect the glucovial cap 22 with the glucovial main body 21 to prevent the glucovial cap 22 from being lost when the glucovial 20 is opened.

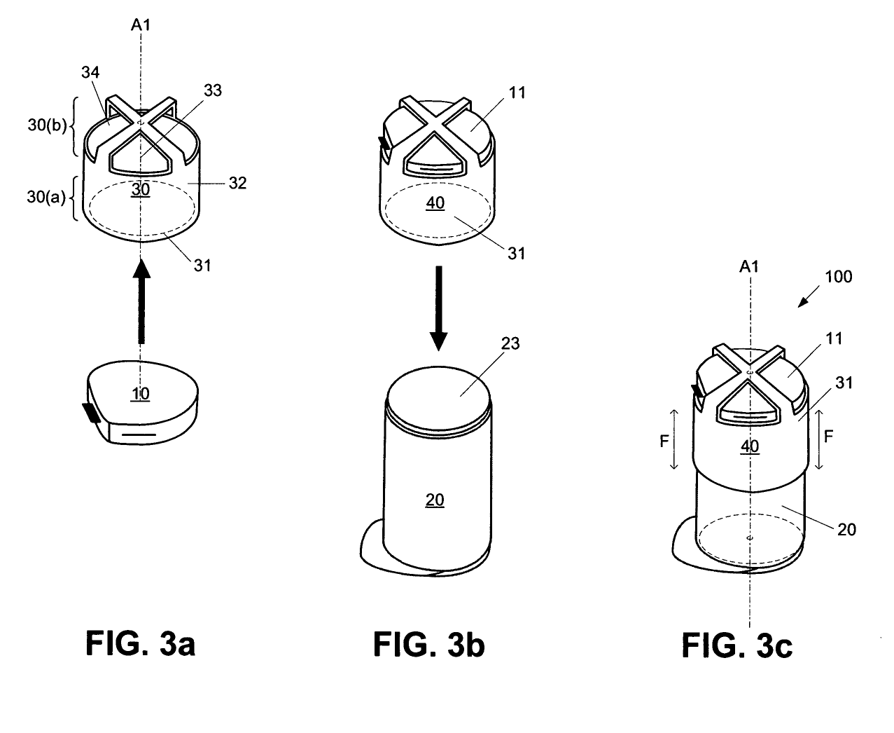

FIG. 3a shows encompassing a glucometer with a glucometer encompassing jacket. More specifically, a glucometer 10 can be inserted into a glucometer encompassing jacket 30 having a lower portion 30a and an upper portion 30b. The glucometer encompassing jacket 30 includes a glucovial-receiving opening 31 surrounded by the wall 32 of the glucometer encompassing jacket 30 in the lower portion 30a of the glucometer encompassing jacket 30. The glucovial-receiving opening 31 can receive the glucometer 10, as shown in FIG. 3a. Although FIG. 3a shows the glucovial-receiving opening 31 having a circular configuration, the glucovial-receiving opening 31 be another shape. For example, the glucovial-receiving opening 31 can be a rectangular, triangular, octagonal or elliptical shape. The lower portion 30a of the glucometer encompassing jacket 30 is composed of a suitably elastic and flexible material. Examples of such materials include flexible polymers, plastics or rubbers. The wall 32 of the glucovial encompassing jacket 30 can be a uniform thickness or can taper along the direction of the central longitudinal axis A1 shown in FIG. 3a.

Glucometer data access opening 33 and glucometer strip access opening 34 are openings in the upper portion 30b of the glucometer encompassing jacket 30 that allow a user to access the glucometer 10 after being encompassed by the glucometer encompassing jacket 30, as shown in FIG. 3a. The glucometer test strip access opening 33 is for receiving test strips from the user. The glucometer data access opening 34 is for providing access to the data output connector 15 of the glucometer 10. There can be a plurality of additional access openings (not shown) in the glucometer encompassing jacket 30. Glucometer test strip access opening 33, glucometer data access opening 34 as well as any other such access openings can be any shape that allows access to the test strip receiving slot 14, the data output connector 15 of the glucometer 10 or to other access points of the glucometer 10. Glucometer test strip access opening 33, glucometer data access opening 34 as well as any other such access openings can also have a rectangular, triangular, octagonal, elliptical or other shape.

The upper portion of the glucometer encompassing jacket 30b can be made of the same material as the lower portion of the glucometer encompassing jacket 30a. Alternatively, the upper portion of the glucometer encompassing jacket 30b can be more wear resistant than the lower portion of the glucometer encompassing jacket 30a so as to resist the increased wear and tear from repeated use of the components of the glucometer, such as connecting and disconnecting the glucometer 10 to a mobile phone via data output connector 15. Examples of wear-resistant materials suitable for the upper portion of the glucometer encompassing jacket 30b include plastics that are harder or more wear resistant than the material of the lower portion of the glucometer encompassing jacket 30a or the same material as the lower portion of the glucometer encompassing jacket 30a that has been hardened and/or toughened by mechanical or chemical processing.

The upper portion of the glucometer encompassing jacket 30b can have a uniform thickness. In the alternative, the upper portion of the glucometer encompassing jacket 30b can taper so as to become thinner in a continuous manner near glucometer data access opening 33, glucometer test strip access opening 34 or other glucometer access openings. Such tapering of the upper portion of the glucometer encompassing jacket 30b provides access to the glucometer data access opening 33, glucometer test strip access opening 34 or other glucometer access openings while also minimizing the amount of material used in the glucovial encompassing jacket 30.

The glucometer 10 is placed in the glucometer encompassing jacket 30 to create the jacketed glucometer assembly 40 shown in FIG. 3b. In the jacketed glucometer assembly 40 the top surface 11 of the glucometer 10 can be partially covered by the glucometer encompassing jacket 30, as shown in FIG. 3b, or the glucometer 10 can be completely covered. Covering the glucometer top surface 11 prevents the glucometer 10 from sliding in a direction away from the glucovial-receiving opening 31.

As shown in FIG. 3c, the jacketed glucometer assembly 40 is brought into contact with the sealed glucovial 20 so that the glucovial top 23 is inserted into the glucovial-receiving opening 31 of the glucometer encompassing jacket 30 and so that the wall 32 of the glucometer encompassing jacket 30 overlaps the glucovial 20 around a substantial portion of the glucovial main body 21. The glucovial top 23 is inserted into the glucovial-receiving opening 31 so as to create the assembly 100 shown in FIG. 3c. The wall 32 of the glucometer encompassing jacket 30 and glucovial main body 21 is such that a substantial portion of the walls of the glucometer encompassing jacket 32 contacts the glucovial main body 21 and such that the jacketed glucometer assembly 40 surrounds the glucovial top 23.

The contact between the wall 32 of the glucometer encompassing jacket 30 and glucovial main body 21, along with the elastic properties of the lower portion of the glucometer encompassing jacket 30a and the wall 32 of the glucometer encompassing jacket 30, will cause the wall 32 of the glucometer encompassing jacket 30 to pinch or exert a force directed from the wall 32 of the glucometer encompassing jacket 30 radially inward with respect to the circumference of the glucovial-receiving opening 31 or towards the central longitudinal axis A1 of glucovial main body 21, as shown in FIG. 3c. The inward force exerted by the wall 32 of the glucometer encompassing jacket 30 on the glucovial main body 21 will cause friction F, indicated by the two arrows in FIG. 3c, between the glucovial 20 and the glucometer encompassing jacket 30. The friction F resists the sliding of the glucometer 10 with respect to the glucovial encompassing jacket 30, that can separate the glucometer 10 from the glucovial 20. Thus, the force or pressure exerted by the wall 32 of the glucometer encompassing jacket 30 constrains the motion of the jacketed glucometer assembly 40 relative to the glucovial 20. The friction F that results from the force or pressure exerted by the wall 32 of the glucometer encompassing jacket 30 holds the glucovial 20 and glucometer 10 together so a user can carry the jacketed glucometer glucovial assembly 100 shown in FIG. 3c as a single unit.

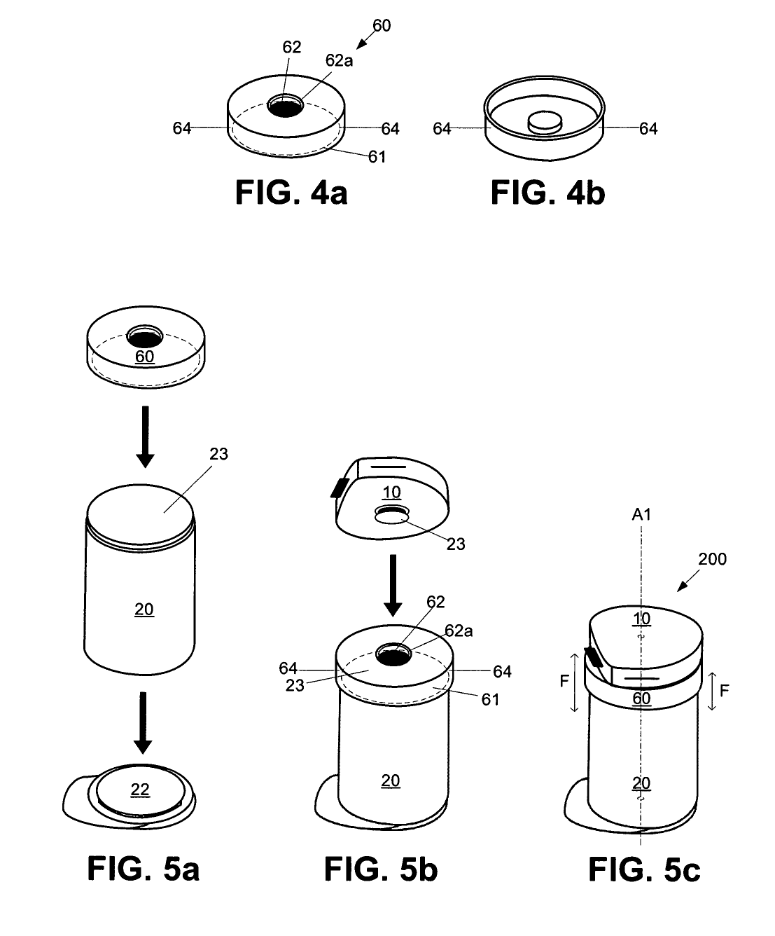

FIG. 4a shows a top view of a glucometer attaching jacket; and FIG. 4b shows a bottom view of the glucometer attaching jacket shown in FIG. 4a. FIG. 5a shows attaching the glucometer attaching jacket shown in FIG. 4a and FIG. 4b to a glucovial; FIG. 5b shows attaching a glucometer to the glucometer attaching jacket attached to the glucovial; and FIG. 5c shows an assembly of a single unit jacketed glucometer glucovial assembly 200.

The glucometer attaching jacket 60 is composed of an elastic and flexible material, such as flexible polymers, plastics or rubbers. The glucometer attaching jacket 60 can be disk shaped, as shown in FIG. 4a, or one of a plurality of other shapes including the shape of a box, a triangular prism or a rectangular prism. The glucometer attaching jacket 60 has a hollow glucovial-receiving opening 61, and a glucometer knob receiving hole 62, as shown in FIG. 4a (top view) and 4b (bottom view). The glucometer knob receiving hole 62 is a part of a reversible fastening mechanism. The lip 62a of the glucometer knob receiving hole 62 shown in FIG. 4a is configured to snap-fasten to the lip 16a of the glucometer fastening knob 16. The lip 62a of the glucometer knob receiving hole 62 can be made of a material that is the same as the rest of the glucometer attaching jacket 60. Alternatively, the lip 62a of the glucometer knob receiving hole 62 can be made of a material that is harder and/or tougher than the rest of the glucometer attaching jacket 60. Examples of a material that is harder and/or tougher include harder plastics or polymers or the same material as the rest of the glucometer attaching jacket 60 after being hardened by a mechanical or chemical process. The wall 64 of the glucometer attaching jacket 60 surround the glucovial receiving opening 61 as shown in FIG. 4a and FIG. 4b.

The glucovial top 23 is inserted into the glucovial-receiving opening 61 to create the assembly shown in FIG. 5b. The overlap of the wall 64 of the glucovial attaching jacket 60 and glucovial main body 21 is such that a substantial portion of the wall 64 of the glucovial attaching jacket 60 contacts the glucovial main body 21 and such that the glucometer attaching jacket 60 covers the glucovial top 23. The glucovial top 23, can be completely covered by the glucometer attaching jacket 60, as shown in FIG. 5b, or partially covered by the glucometer attaching jacket 60.

The glucometer 10 is positioned on the glucometer attaching jacket 60, as shown in FIG. 5b, such that the glucometer fastening knob 16 of the glucometer 10 is reversibly affixed in the glucometer fastening knob receiving hole 62 by the lip 62a. Alternatively, the glucometer fastening knob receiving hole 62 can contain one of a plurality of other mechanisms, such as a latch or spring mechanism (not shown), that reversibly fasten the glucometer fastening knob 16 in the glucometer fastening knob receiving hole 62. The fastening of the glucometer fastening knob 16 into the glucometer fastening knob receiving hole 62 creates the assembly 200 shown in FIG. 5c.

In the assembly 200 shown in FIG. 5c, the contact between the wall 64 of the glucovial attaching jacket 60 and glucovial main body 21, along with the elastic properties of the glucovial attaching jacket 60 and the wall 64 of the glucovial attaching jacket 60, will cause the wall 64 of the glucovial attaching jacket 60 to pinch or exert a force directed from the wall 64 of the glucovial attaching jacket 60 radially inward with respect to the circumference of the glucovial-receiving opening 31 or towards the central longitudinal axis A1 of glucovial main body 21, as shown in FIG. 5c. The inward force exerted by the walls of the glucovial attaching jacket 64 on the glucovial main body 21 will give rise to friction F, indicated by the two arrows in FIG. 5c, between the glucovial 20 and the glucovial attaching jacket 60. The friction F resists the sliding of the glucometer 10 with respect to the glucovial attaching jacket 60 that can separate the glucometer 10 from the glucovial 20. Thus, the force or pressure exerted by the wall 64 of the glucovial attaching jacket 60 constrains the motion of the glucometer 10 and glucometer attaching jacket 60 relative to the glucovial 20. The friction F that results from the force or pressure exerted by the wall 64 of the glucometer attaching jacket 60 holding the glucovial 20 and glucometer 10 together so that a user can carry the jacketed glucometer glucovial assembly 200 shown in FIG. 5c as a single unit.

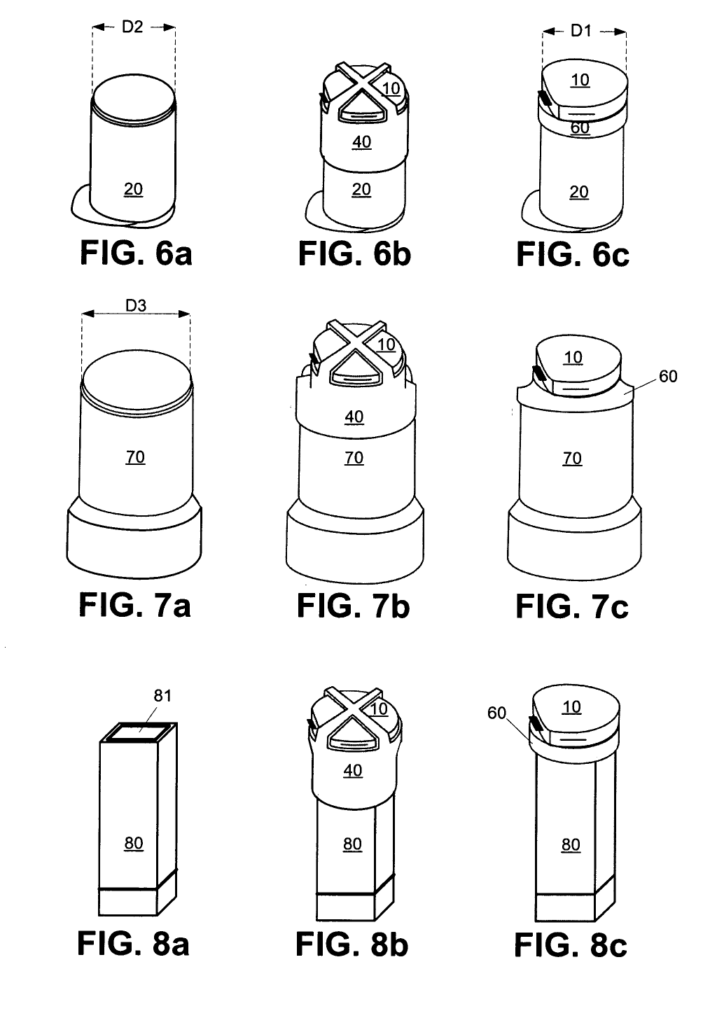

The glucometer connector system is “universal” in the sense that the glucometer connector system attaches a glucometer to any one of a plurality of glucovials provided by a variety of manufacturers. Further, the universal glucometer connector system attaches a glucometer to glucovials that have any one of a plurality of configurations, shapes and sizes. Several examples of the different types of glucovials that can be used with the system are illustrated in FIGS. 6-8.

FIG. 6a shows a cylindrical glucovial 20 with a circular cross-section having a diameter D2 that is approximately equal to the width D1 of the glucometer 10. FIG. 6b shows a jacketed glucometer assembly 40, in which a glucometer 10 is encompassed in a glucometer encompassing jacket 30, which is attached to the cylindrical glucovial 20 shown in FIG. 6a. FIG. 6c shows a glucometer 10 attached to a glucometer attaching jacket 60, which is attached to the cylindrical glucovial 20 shown in FIG. 6a. The wall 32 of the glucometer encompassing jacket 30 and the wall 64 of the glucometer attaching jacket 60, slide over the top 23 of the glucovial 20 and elastically adhere around the glucovial 20 in the configuration illustrated in FIGS. 6b and 6c, respectively.

FIG. 7a shows an oversized cylindrical glucovial 70 with a circular cross-section having a diameter D3 that is substantially larger than the width D1 of a glucometer 10. FIG. 7b shows a jacketed glucometer assembly 40, in which a glucometer 10 is encompassed in a glucometer encompassing jacket 30, which is attached to the oversized cylindrical glucovial 70 shown in FIG. 7a. FIG. 7c shows a glucometer 10 attached to a glucometer attaching jacket 60, which is attached to the oversized cylindrical glucovial 70 shown in FIG. 7a. The wall 32 of the glucometer encompassing jacket 30 and the wall 64 of the glucometer attaching jacket 60, slide over the walls of the oversized cylindrical glucovial 70 and elastically adhere around the oversized cylindrical glucovial 70 in the configuration illustrated in FIGS. 7b and 7c, respectively.

Because the wall 32 of the glucometer encompassing jacket 30 and the wall 64 of the glucometer attaching jacket 60 are made of elastic material, they stretch to fit around the oversized cylindrical glucovial 70 despite the increased width D3 over that of the cylindrical glucovial 20 with a circular cross-section having a diameter D2 that is approximately equal to the width D1 of the glucometer 10. The upper size limit for the glucovial diameter fit by the wall 32 of the glucometer encompassing jacket 30 and the wall 64 of the glucometer attaching jacket 64 is determined by the elastic properties of the materials used to make them.

FIG. 8a shows a square glucovial 80 with a square cross-section and FIG. 8b shows a jacketed glucometer assembly 40, in which a glucometer 10 is encompassed in a glucometer encompassing jacket 30, which is attached to the square glucovial 80 shown in FIG. 8a. FIG. 8c shows a glucometer 10 affixed to a glucometer attaching jacket 60, which is attached to the glucovial 80 shown in FIG. 8a. The wall 32 of the glucometer encompassing jacket 30 and the wall 64 of the glucometer attaching jacket 60 are slid over the top 81 of the square glucovial 80 as shown in FIGS. 8b and 8c, respectively. Additionally, the wall 32 of the glucometer encompassing jacket 30 and the wall 64 of the glucometer attaching jacket 60 slide over the top of glucovials (not shown) with non-square and non-circularly shaped cross-sections, since the wall 32 and the wall 64 have elastic properties. These include glucovials with triangular cross sections as well as non-cylindrically shaped glucovials such as spherically shaped glucovials.

The capability of the wall 32 of the glucometer encompassing jacket 30 and the wall 64 of the glucometer attaching jacket 60 to stretch onto glucovials of different designs, sizes and configurations is one of the advantages of this invention. Fitting multiple glucovial designs, sizes and configurations gives the user the flexibility to chose the glucovial/test strip combination that best suits his/her needs, even as those needs change. For example, the user can choose another glucovial/test strip combination if the currently used combination suddenly becomes commercially unavailable. The user may also wish to switch from using a certain glucovial/test strip combination to another that is more economical. With embodiments of the invention, the user can purchase and use glucovials provided by different manufacturers having different shapes, using entirely different configurations for opening, closing and test strip storing as well as glucovials that are of different sizes without replacing the glucometer.

The flexibility to change glucovials without replacing the glucometer provided by embodiments of this invention also allows the user to use glucovials for special or temporary needs. Examples of glucovials designed for special or temporary needs include glucovials reinforced for use in harsh outdoor environments, such as those used during camping or hiking excursions, waterproofed glucovials for use in water-related leisure activities or waterborne travel, or large glucovials that contain a supply of test strips sufficient to supply the user during an extended vacation or business trip. While glucovials designed for special or temporary needs are appropriate for these specific occasions, their designs can make such glucovials less convenient for daily use. Thus, the glucovial system in embodiments of the invention allows the user to switch between glucovials designed for special or temporary needs, and more standard glucovials without replacing the glucometer.

With embodiments of the invention, the user can replace glucovials that have become faulty due to wear, damage or manufacturing defect. For example, glucovials having a defective flange between the glucovial main body 21 and the glucovial cap 22 can be replaced. Defective flanges can be caused by dropping the open glucovial 20 on a hard surface, using the glucovial 20 for unintended purposes or by wear from repeated use. A defective flange can leave the test strips stored inside the glucovial main body 21 exposed to ambient contaminants and humidity that could lead to potentially dangerously erroneous diagnostic results.

The glucovial 20 can also be replaced if the desiccant naturally decreases in effectiveness with age. Typically, the lifetime of the desiccant can be considerably shorter than the time for the glucovial to wear out, break apart or lose structural integrity. Embodiments of the invention allow the user to simply remove the glucovial with the ineffective desiccant from the system and replace it with a new glucovial with a fresh desiccant. This capability can extend the lifetime and usefulness of the glucometer, save resources and make long term monitoring more accurate.

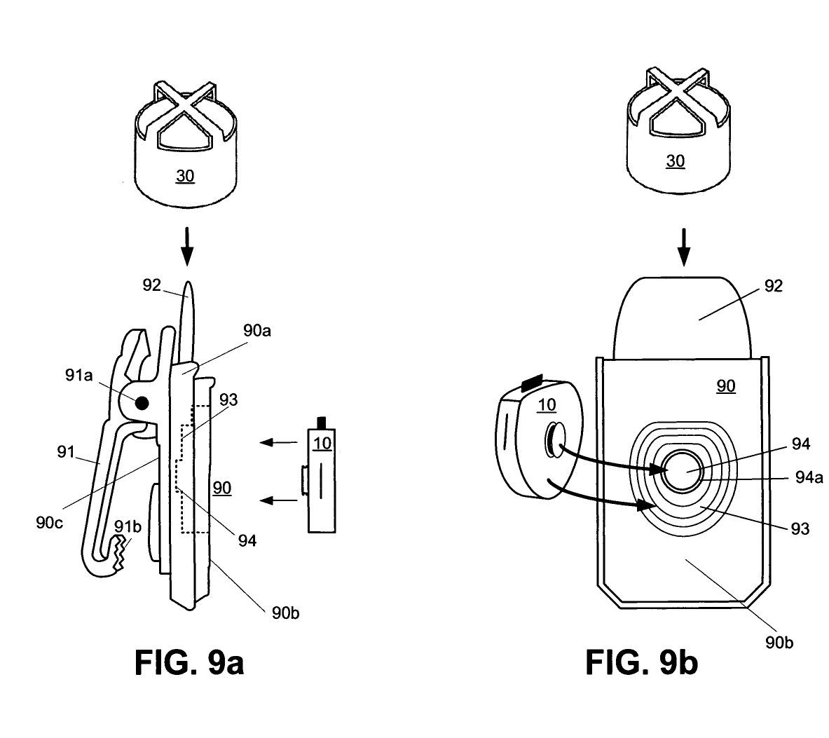

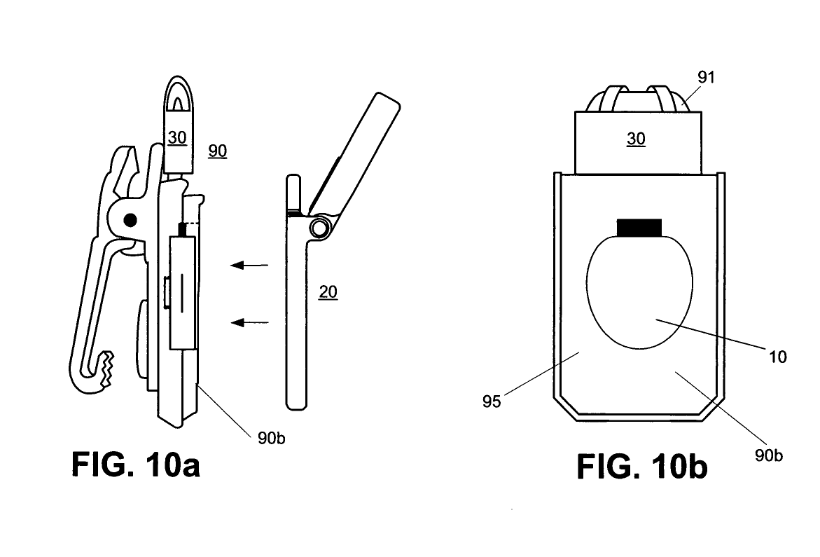

FIG. 9a shows a side view of storing a glucometer encompassing jacket 30 and glucometer 10 in a holster 90. As shown in FIG. 9a, the design of the holster 90 is based on that of a mobile phone holster having a top portion 90a, an interior face 90b and an exterior face 90c. FIG. 9b shows a front view of storing a glucometer encompassing jacket 30 and glucometer 10 in a holster 90. Although only the glucometer encompassing jacket 30 is illustrated in FIG. 10, the holster 90 can be adapted to store any jacket in accordance with embodiments of this invention.

The holster 90 includes at least one holster clip 91 that fastens to an article of clothing such as a shirt pocket or a belt. The holster clip 91 can be affixed to the exterior face 90c of the holster 90 via a hinge 91a, as shown in FIG. 9a. Alternatively, The holster clip 91 can be affixed the exterior face 90c of the holster 90 via a spring mechanism (not shown) and/or an adjustable latch (not shown). The holster clip 91 can have teeth 91b, as shown in FIG. 9a, for gripping an article of clothing.

The holster 90 contains at least one post 92 disposed on the top portion 90a of the holster 90, as shown in FIG. 9a. The post 92 secures the glucometer encompassing jacket 30 while not in use. The post 92 can further have a strap, snap, latch or spring lock (not shown) to hold the glucometer encompassing jacket 30 in place.

As shown in FIG. 9a, the holster 90 also contains at least one glucometer storing recessed portion 93 on the interior face 90b of the holster 90 for storing the glucometer 10. This glucometer storing recessed portion 93 can be ribbed, as shown in FIG. 9a, to match contours of the glucometer 10. Alternatively, the glucometer storing recessed portion 93 may be flat. The glucometer storing recessed portion 93 of the holster 90 can contain a latch or spring mechanism (not shown) that locks the glucovial 10 in place. Alternatively, it can contain a plastic lip (not shown) that snaps the glucometer 10 into place. The glucometer storing recessed portion 93 of the holster 90 can also contain a further glucometer fastening knob accepting recessed portion 94, shown in FIG. 2a, for securing the glucometer 10 by reversibly fastening the glucometer fastening knob 16. The glucometer fastening knob accepting recessed portion 94 can contain any number of locking mechanisms, such as the plastic lip 94a shown in FIG. 9b for snap-fastening the lip 16a of the glucometer fastening knob 16 into place. Alternatively, the glucometer fastening knob accepting recessed portion 94 can also contain one of a plurality of other mechanisms (not shown), such as simple latches or springs, for reversibly fastening the glucometer fastening knob 16 into place. Both FIGS. 9a and 9b show how the glucometer encompassing jacket 30 of embodiments of the invention placed onto the post 92 and the glucometer 10 in the glucometer storing recessed portion 93 of the holster 90. FIGS. 9a and 9b show this from the side and front views, respectively.

FIG. 10a shows a side view of a glucometer encompassing jacket 30 and glucometer 10 stored in a holster 90 and the placing of a mobile phone 20 in the holster 90. The mobile phone 20 is placed on the interior face 90b of the holster 90 so that it covers the glucometer 10, which is fitted into the glucometer storing recessed portion 93 of the holster 90. FIG. 10b shows a front view of a glucometer encompassing jacket 30 and glucometer 10 stored in a holster 90. Although only the glucometer encompassing jacket 30 is illustrated in FIG. 10, the holster 90 can be adapted to store any jacket in any of the embodiments of this invention. FIG. 10a shows that the glucometer 10 is flush with the interior face 90b of the holster 90 when affixed into the glucometer storing recessed portion 93 via fixing the glucometer fastening knob 16 to 94. However, the glucometer storing recessed portion 93 can also be sufficiently shallow so that the inserted glucometer 10 is not flush with the interior face 90b of the holster 90.

Once the glucometer 10 is placed in the glucometer storing recessed portion 93, the mobile phone 20 can be placed over the glucometer on the interior face 90b of the holster 9, as shown in FIG. 10a. The mobile phone 20 is affixed to the holster 90. Once the mobile phone 20 is affixed to the interior face 90b of the holster 90, the entire apparatus can be carried as a single unit (not shown). The construction of the glucometer storing recessed portion 93 and glucometer fastening knob accepting recessed portion 94 can be such that the assembly in FIG. 10a and 10b after attachment of the mobile phone 20 is not bulkier than if the mobile phone 20 were stored on the holster by itself. In addition, both the glucometer 10 and the glucometer encompassing jacket 30 can be stored securely on the holster 90 in such a way as to prevent loss.

The holster 90 and the assembly shown in FIG. 10 keeps the universal glucometer connector system as accessible as any cellular phone. In the case of use, for example, the user could easily remove the mobile phone 20 and glucometer 10 from their fixed positions illustrated in FIG. 10a, connect the mobile phone 20 to the glucometer 10 via the data output connector 15, use the system to obtain a blood glucose concentration measurement and then use the mobile phone 20 to send the data to a medical professional for further analysis and/or diagnosis.

It will be apparent to those skilled in the art that various modifications and variations can be made in the universal glucometer/glucovial connector of embodiments of the invention without departing from the spirit or scope of the invention. Thus, it is intended that embodiments of the invention cover the modifications and variations of this invention provided they come within the scope of the appended claims and their equivalents.

What is claimed is:

1. A universal glucometer connector system comprising:
a glucometer for measuring blood glucose concentration, and having top, bottom and side surfaces;
a jacket for retaining the glucometer;
a first opening in the jacket for receiving a glucovial that can be one of a plurality of shapes and sizes.

2. The universal glucometer connector system of claim 1, wherein the jacket is attachable to the glucovial via an attachment mechanism.

3. The universal glucometer connector system of claim 1, wherein the glucometer is configured to communicate measured blood glucose concentration to a mobile phone.

4. The universal glucometer connector system of claim 1, further comprising a glucovial having a container for receiving test strips and a cap for sealing the container, wherein the first opening of the jacket encompasses an end of the container opposite to another end of the container to which the cap attaches.

5. The universal glucometer connector system of claim 3, wherein the jacket includes an elastic material.

6. The universal glucometer connector system of claim 1, wherein a portion of the side surface of the glucometer is exposed by a second opening in the jacket such that the glucometer can receive test strips.

7. The universal glucometer connector system of claim 1, wherein the jacket encompasses the top surface of the glucometer.

8. The universal glucometer connector system of claim 7, wherein the jacket encompasses the side surface of the glucometer.

9. A universal glucometer connector system comprising:
a glucometer for measuring blood glucose concentration;
a glucovial having a container for receiving test strips and a cap for sealing the container;
a jacket retaining the glucometer within the jacket;
a first opening in the jacket such that the glucometer can receive test strips; and
a second opening in the jacket that surrounds the glucovial.

10. The universal glucometer connector system of claim 9, wherein a portion of the side surface of the glucometer is exposed by the first opening of the jacket.

11. The universal glucometer connector system of claim 9, wherein the jacket includes an elastic material.

12. The universal glucometer connector system of claim 11, wherein the jacket holds the glucovial by exerting a pressure towards a central longitudinal axis of the glucovial such that the pressure creates friction between the jacket and the glucovial.

13. A universal glucometer/glucovial connector system comprising:
a glucometer for measuring glucose concentration of blood;
a mobile phone for receiving signals from the glucometer, interpreting the signals and displaying the data including the measured glucose concentration; and
a jacket for receiving a glucovial that can be one of a plurality of shapes and sizes.

14. The universal glucometer connector system of claim 13, wherein the glucometer is connected to the mobile phone using local wireless network.

15. The universal glucometer connector system of claim 13, wherein the glucometer is connected to the mobile phone through a serial connection.

16. The universal glucometer connector system of claim 13, further comprising a glucovial having a container for receiving test strips and a cap for sealing the container, wherein the second opening of the jacket encompasses an end of the container opposite to another end of the container for receiving the cap.

17. The universal glucometer connector system of claim 13, wherein a portion of the side surface of the glucometer is exposed by the first opening of the jacket.

18. The universal glucometer connector system of claim 17 wherein the jacket includes an elastic material.

19. The universal glucometer connector system of claim 18 wherein the jacket holds the glucovial and glucometer together by exerting a pressure towards a central longitudinal axis of the glucovial such that the pressure creates friction between the jacket and the glucovial.

20. A storage system for a universal glucometer connector system comprising:
a glucometer for measuring glucose concentration of blood;
a holster that has a top portion and a main-body portion, wherein the main-body portion includes:
an outward face having a recessed portion in which the glucometer can be reversibly fixed and a second portion where the mobile phone can be reversibly fixed;
an inward face opposite to the outward face and having a clip that allows the holster to be fixed to an article of clothing; and
the top portion extends above the clip to form a post on which a jacket for receiving a glucovial can be stored.
